(19) United States
(12) Patent Application Publication
(10) Pub. No.: US 2011/0064675 A1
(43) Pub. Date: Mar. 17, 2011

(54) ELEMENTAL IRON NANOPARTICLES

(75) Inventors: Constantinos G. Hadjipanayis, Decatur, GA (US); George C. Hadjipanayis, Wilmington, DE (US); Michael J. Bonder, Livingston, NJ (US)

(73) Assignees: EMORY UNIVERSITY, Atlanta, GA (US); UNIVERSITY OF DELAWARE, Newark, DE (US)

(21) Appl. No.: 12/887,689

(22) Filed: Sep. 22, 2010

Related U.S. Application Data
(63) Continuation of application No. PCT/US2009/038112, filed on Mar. 24, 2009.
(60) Provisional application No. 61/039,293, filed on Mar. 25, 2008.

(51) Int. Cl. A61K 49/14; A61K 49/16; A61K 9/50; A61K 33/26; A61K 39/395; A61P 35/00

(52) U.S. Cl. 424/9.34; 424/490; 424/646; 424/158.1; 424/172.1

(57) ABSTRACT

Synthesis of iron nanoparticles with a substantially unoxidized iron core and a biocompatible coating is described. The nanoparticles are formed by reacting an iron salt solution with a reducing agent in a substantially oxygen-free environment and exposing the formed iron particles to a biocompatible coating agent in a substantially oxygen-free environment to form coated iron particles. An average diameter of the coated iron particles is between 5 nm and 25 nm. The biocompatible coating can functionalized with cell-specific agents for use as diagnostic and therapeutic agents.

### PRIORITY CLAIM

This application is a continuation and claims priority to PCT application no. PCT/US2009/038112 filed Mar. 24, 2009 and claims priority to provisional application no. 61/039,293 filed on Mar. 25, 2008. All documents disclosed herein are incorporated by reference.

### BACKGROUND

Nanoparticles with an iron oxide core are currently used in biomedical applications. These iron oxide nanoparticles are tolerated by the human body, degrade over time, and can be coated with a biocompatible substance that can be further functionalized to allow targeted delivery and sustained release. The iron oxide core is detectable by magnetic resonance imaging (MRI) devices, though due to the oxidation in the core there are limitations on detection.

### SUMMARY

Substantially unoxidized iron nanoparticles that are manipulable by magnetic fields, methods for making the substantially unoxidized iron nanoparticles, and methods for using the substantially unoxidized iron nanoparticles are provided. For example, the substantially unoxidized iron nanoparticles include a substantially unoxidized iron core and a biocompatible coating. The biocompatible coating substantially prevents oxidation of the unoxidized iron core. The average diameter of such particles is between 5 nm and 25 nm.

A method for making these particles includes reacting an iron salt solution with a reducing agent in a substantially oxygen-free environment to form the substantially unoxidized iron nanoparticles. Then, once the particles have formed, exposing the formed substantially unoxidized iron nanoparticles to a biocompatible coating agent in a substantially oxygen-free environment to form coated substantially unoxidized iron nanoparticles. Substantially unoxidized iron nanoparticles made by this method are substantially free of iron oxide and the average diameter of the coated iron particles is between 5 nm and 25 nm.

An apparatus for making the substantially unoxidized iron nanoparticles includes a first reservoir containing a reducing agent solution, a reaction fitting in fluid connection with the first reservoir, a second reservoir containing an iron salt solution in fluid connection with the reaction fitting, a first fluid flow regulator operative to control fluid flow between the second reservoir and the reaction fitting, and a particle collection reservoir in fluid connection with the reaction fitting. The apparatus also includes a second fluid flow regulator operative to control fluid flow between the reaction fitting and the particle collection reservoir and a fluid collection reservoir in fluid connection with the reaction fitting. The first and second fluid flow regulators are opened to initiate a reaction between the reducing agent and the iron salt solution.

Finally, methods for using the substantially unoxidized iron nanoparticles include methods for detecting specific target cells, methods for effecting cell lysis of a target cell, methods for directing magnetic particles to a particular area of a patient, and methods for effecting cell movement. These methods each involve providing substantially unoxidized iron nanoparticles to a patient then manipulating the substantially unoxidized iron nanoparticles using a magnetic field proximate the patient. Such manipulations can include imaging the particles, changing the position of the particle, and/or increasing the temperature of the particles.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic view of a frequency modulated flow reactor.

FIG. 2 is a schematic view of a reaction fitting.

FIG. 3A is schematic of a cPEG coated iron nanoparticle.

FIG. 3B is a low magnification bright field TEM image of coated iron nanoparticles.

FIG. 3C is a TEM image of a single iron nanoparticle.

FIG. 3D is a selected area electron diffraction pattern for a single iron nanoparticle.

FIG. 4 is a graph showing magnetization curves for iron nanoparticles and oxidized-iron nanoparticles.

FIG. 5 is a graph showing change in temperature over time for iron nanoparticles and oxidized-iron nanoparticles excited in a 4 Oe 500 kHz magnetic field.

FIG. 6 is graph showing MRI signal decay over time for iron nanoparticles and oxidized-iron nanoparticles.

### DETAILED DESCRIPTION

Particles manipulable by magnetic fields, methods for making the particles, and methods for using the particles are disclosed herein. These particles are useful, for example, in magnetic imaging and other techniques involving the manipulation of magnetic fields. The particles include a substantially unoxidized iron core and a biocompatible coating. The biocompatible coating substantially prevents oxidation of the unoxidized iron core. The biocompatible coating is capable of being functionalized with biologically active molecules.

The core of the iron nanoparticles described herein is unoxidized elemental iron. The substantially unoxidized cores are highly manipulable by magnetic fields, and have a high saturation magnetization (Ms). In fact, the Ms for the unoxidized cores is an order of magnitude greater than that of a similarly sized iron oxide core. This high Ms value allows for effective contrast enhancement in magnetic resonance imaging. When in a magnetic field, the iron nanoparticles demonstrate a superparamagnetic effect that limits agglomeration and improves stability. The substantially unoxidized iron cores have an average diameter between 2 nm and 30 nm, between 5 nm and 25 nm, between 6 nm and 20 nm, between 7 nm and 15 nm, between 8 nm and 12 nm, and 10 nm. The average diameter of the particles also can be less than 30 nm, less than 25 nm, less than 20 nm, less than 15 nm, less than 12 nm, and less than 11 nm. The phrase average diameter as used herein is intended to mean the average diameter of a set of particles.

The iron nanoparticles described herein are coated with a biocompatible coating to inhibit oxidation of the substantially unoxidized iron core. The coating is any biocompatible substance that bonds to the iron in the nanoparticle or coats a particle such that oxidation is inhibited. Particular biocompatible coating materials can be tailored to address issues of reactivity in an aqueous environment, biocompatibility, solubility, and/or stability. Different coatings can be chosen for different properties such as biocompatibility, solubility, hydrophobicity, hydrophilicity, melting point, and degradation over time. Examples of coatings include, polysaccharide, dextran, polyvinyl methacrylate, polymethyl methacrylate, oleic acid, polyethylene glycol, carboxylated polyethylene glycol (cPEG), methoxy PEG, amphiphilic triblock polymers, and mixtures thereof. A coating can be selected based on its ability to be functionalized. For example a coating molecule can be selected based on its ability to bind a specific drug, dye, or viral construct. Coating combinations can optimize multiple properties, e.g., nanoparticles coated with a combination of amphiphilic triblock polymers and cPEG can both improve solubility in aqueous solutions and be functionalized. The biocompatible coatings on the unoxidized iron cores have an average thickness of between 0.5 nm and 4 nm, between 1 nm and 3 nm, and 2 nm. The average thickness of the biocompatible coatings also can be less than 5 nm, less than 4 nm, less than 3 nm, less than 2 nm, and less than 1 nm.

Functionalization of the biocompatible coating can be by covalent binding or ionic interaction. For example, a cell-specific agent can be covalently bonded to a biocompatible coating through a condensation reaction. In the case of cPEG, the cell-specific agent undergoes a condensation reaction with the terminal carboxy group to form the functionalized nanoparticle. The cell-specific agent can be, for example, a cancer-cell-specific agent or a vascular-cell-specific agent. Functionalization of biocompatible coatings with cancer-cell-specific agents, polypeptides, antibodies, and cancer therapeutics allows cancer cell detection as well as targeted therapy that can be imaged with MRI. For example, proteins such as IL-4 and IL-13 can be conjugated to the coated nanoparticles for cancer cell detection. Targeted therapy is achieved using iron nanoparticles with biocompatible coatings conjugated to antibodies used against various target cells, such as brain, lung, breast, renal, and pancreatic cancer cells, including cancer stem cells.

Such antibodies include, for example, herceptin, tarceva, erbitux, antibodies to VEGF and VEGF receptors, antibodies to PDGF receptors, antibodies to EGF and truncated vIII EGF receptors, antibodies to IL-13 receptors, antibodies to IL-3 receptors, antibodies to IL-4 receptors, antibodies to IL-12 receptors, antibodies to chemokine receptor CXCR4, antibodies to TGF beta receptors, antibodies to TGF alpha receptors, antibodies to IGF-1 receptors (insulin like growth factor), antibodies to TRAIL, antibodies to Her-1 receptors, antibodies to estrogen receptors, antibodies to Her-2 receptors, antibodies to tenascin, antibodies to nestin, antibodies to HIF-α, antibodies to p53, antibodies to MDM2, antibodies to Rb, antibodies to neurofibromin, antibodies to cMYC, antibodies to nMYC, antibodies to Ras, antibodies to CMET, antibodies to PTEN, antibodies to Akt, antibodies to Sonic Hedgehog (SHH), antibodies to CD20, antibodies to CD31, and antibodies to CD34. Antibodies for cancer stem cell targeting include, for example, antibodies to CD133, CD44, CD24, CD19, CD34, BMP (bone-morphogenic protein) receptors, and musashi. Further cell specific agents include, for example, viral proteins and viruses (e.g., Herpes simples virus 1, Herpes simples virus 2, adenovirus, adeno-associated virus, haemophilus influenza virus, and lentiviruses such as human immunodeficiency virus (HIV)).

Overall, the coated and/or functionalized iron nanoparticles described herein have an average diameter between 2 nm and 30 nm, between 5 nm and 25 nm, between 6 nm and 20 nm, between 7 nm and 15 nm, between 8 nm and 12 nm, and 10 nm. The average diameter of the coated and/or functionalized iron nanoparticles also can be less than 35 nm, less than 30 nm, less than 25 nm, less than 20 nm, less than 15 nm, less than 12 nm, and less than 11 nm. Confirmation of the average diameters of a set of nanoparticles can be obtained, for example, by the use of electron microscopy.

A method for forming these iron nanoparticles is to reduce an iron salt with a reducing agent in the substantial absence of oxygen, then to expose the particles formed to the biocompatible coating material. The iron salt can be, for example, iron chloride or iron sulfate. Examples of reducing agents include sodium borohydride, potassium borohydride, lithium borohydride, ammonia, and mixtures thereof. Iron nanoparticles can be formed, for example, by the reduction of iron chloride with sodium borohydride, as shown in equation I (unbalanced).

(FeCl2+EtOH)+(NaBH4+H2O(deionized))→Fe(B)+NaCl+H2O+EtOH+H2 (1)

The reduction of the ferrous chloride produces iron nanoparticles with the incorporation of interstitial boron that results from the reduction reaction. An excess of iron salt can be used to maximize the Fe content, e.g., a 1.5-2.5 (or 1.7-2) molar excess of iron salt to reducing agent. The average diameter of the particles formed as well as the distribution of diameters are determined by parameters such as, for example, contact time of the reagents and agitation of the particles during and after formation. The iron nanoparticle cores thus formed can be coated in a solution substantially free of oxygen to further inhibit oxidation of the iron cores. Coating occurs through bonding of a biocompatible substance to the surface of the iron nanoparticle and/or interactions between the molecules forming the coating. The reaction between a biocompatible coating such as a carboxyl group on a cPEG molecule and an iron nanoparticle surface can incorporate a slight oxide layer that facilitates biocompatibility and prevents excessive oxidation of the nanoparticles. Iron nanoparticles made by this method are thought to have a slight oxidation layer that varies between nanoparticles and is less than a few percent by weight of an iron nanoparticle, e.g., less than 2 percent by weight. The efficacy of the particles depends on the formation of an unoxidized iron core and the presence of iron oxide at the surface is tolerable.

One method for making coated iron nanoparticles includes reducing an iron salt with a strong reducing agent and exposing the resulting iron nanoparticle cores to a biocompatible coating material in the presence of a biocompatible coating in a frequency modulated micro-burst flow reactor. The exposure of the iron nanoparticle cores to the biocompatible coating material can also be considered to quench the iron nanoparticle core formation, as the iron nanoparticle cores will no longer increase in size once the biocompatible coating is in place. In the reactor, the reduction reaction occurs and the resulting iron nanoparticle cores are exposed to biocompatible coating material in a substantially oxygen-free environment.

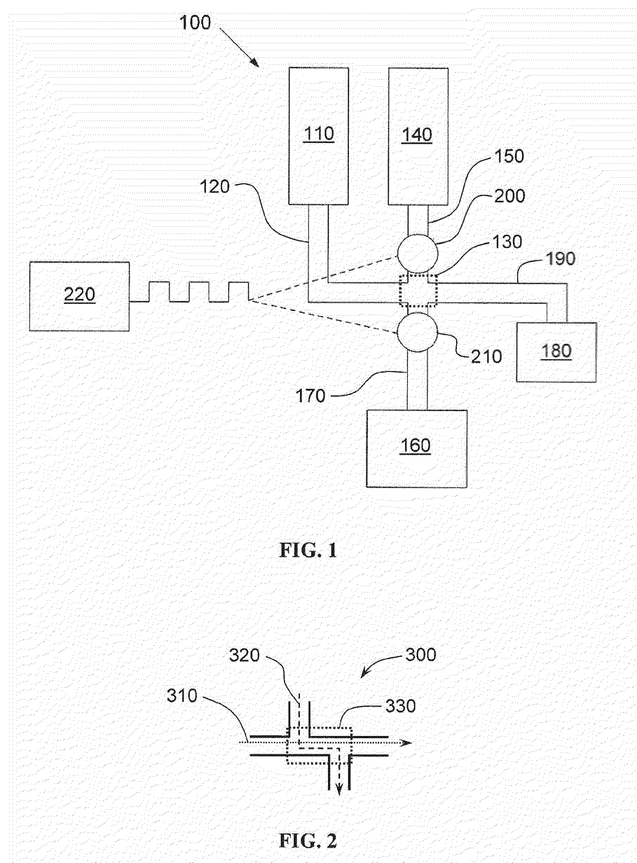

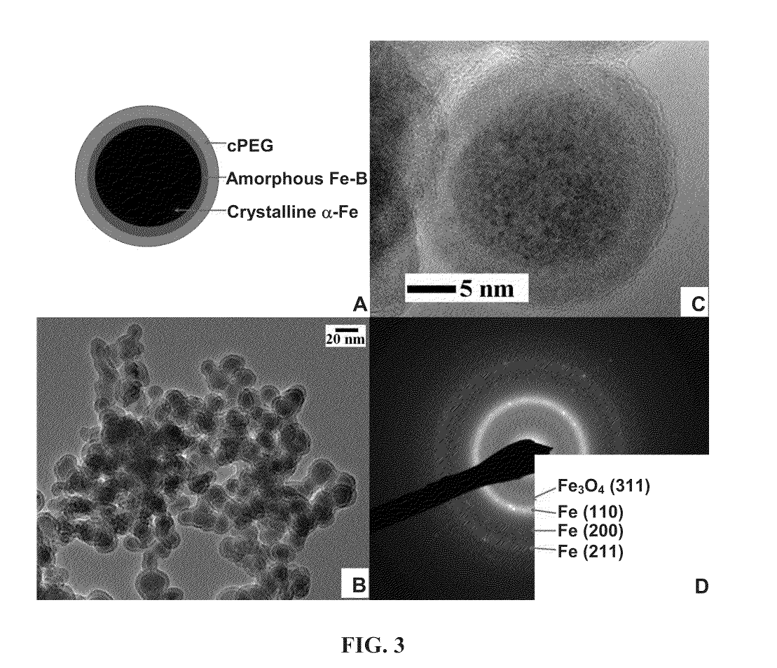

FIG. 1 depicts a schematic view of a frequency modulated flow reactor 100. The frequency modulated flow reactor 100 includes a first reservoir 110 fluidly connected by a first fluid conduit 120 to a reaction fitting 130. The reaction fitting 130 is also fluidly connected to a second reservoir 140 by a second fluid conduit 150. The reaction fitting 130 is further fluidly connected to a particle connection reservoir 160 by a third fluid conduit 170. The reaction fitting 130 is additionally fluidly connected to a fluid collection reservoir 180 by a fourth fluid conduit 190. The flow of fluid from the second reservoir 140 along second fluid conduit 150 is controlled by a first fluid flow regulator 200 and the flow of fluid along the third fluid conduit 170 to the particle collection reservoir 160 is controlled by a second fluid flow 210. The first fluid flow regulator 200 and second fluid flow regulator 210 are frequency controlled solenoid valves that are controlled by a controller 220. As shown in FIG. 1, the flow paths within the reaction fitting 130 are substantially perpendicular. Flow paths within a reaction fitting, including the angle the flow paths intersect, can be altered to impact, for example, the amount of time two fluids come into contact. FIG. 2 shows another reaction fitting 300 within a first flow path 310, a second flow path 320, and a combined flow path region 330.

Fluid flow along any of the fluid conduits of frequency modulated flow reactor 100 can be controlled by manually or automatically controlled flow regulators. For example, a flow regulator can be a solenoid valve. The controller 220 is operatively interconnected with the first fluid flow regulator 130 and the second fluid flow regulator 210. The controller 220 has hardware and/or software configured for operation of these components 130 and 210, and may comprise any suitable programmable logic controller or other control device, or combination of control devices, that is programmed or otherwise configured to perform as recited in the claims.

In the reactor 100, a reducing agent flows continuously from the first reservoir 110 and through the reaction fitting 130 to react with an iron salt solution flowing from the second reservoir 140. Nanoparticles are formed within the reaction fitting 130 within the region in which the flow paths combine. Flow of the iron salt solution from the second reservoir 140 is modulated by a fluid flow regulator 200 pulsed at a frequency generated by the controller 220. Variations in modulation frequency, pressure of reagents, cross-sectional area of the reaction conduit, and contact angle of the reagents affect reaction time, particle size, and particle size distribution. The modulation frequency can be in a range of 1 Hz to 100 Hz, 1 Hz to 40 Hz, or 5 Hz. Alternatively, the modulation frequency can be less than 200 Hz, 100 Hz, 80 Hz, 60 Hz, 40 Hz, 20 Hz, 10 Hz, or 5 Hz.

In the frequency modulated flow reactor 100, when the iron salt solution and reducing agent come into contact, nanoparticles are formed that fall vertically through the third fluid flow conduit 170 and the second fluid flow regulator 210 which is pulsed at a frequency generated by the controller 220. The particles formed are thought to fall into the third fluid flow conduit 170 due to a combination of gravitational forces and fluid flow. In some implementations, regulators 200 and 210 are modulated at substantially the same frequency by a common signal generated by the controller 220, such that the regulators are opened and closed at substantially the same time. Nanoparticles that form in the reaction fall through the third fluid flow conduit 170 into the particle collection reservoir 160. The particle collection reservoir 160 contains a solution capable of coating nanoparticles that fall into the reservoir 160 through the third fluid flow conduit 170. The particle collection reservoir 160 can contain, for example, ethyl alcohol and cPEG. The fluid path through the reaction fitting 130, the third fluid flow conduit 170, and the particle collection reservoir 160 is substantially free of oxygen. The particle collection reservoir 160 can be sonicated during the collection process to facilitate coating of the nanoparticles as the coating solution reacts with the substantially unoxidized surface of the nanoparticles. Coating the nanoparticles as described substantially prevents oxidization of the unoxidized iron core. Coated nanoparticles created using the frequency modulated flow reactor 100 can be stably suspended in water.

Excess reducing agent and any particles that do not fall into the third fluid flow conduit 170 and the particle collection reservoir 160, flow through the fourth fluid flow conduit 190 and into the fluid collection reservoir 180. The residual solution and particles collected in the fluid collection reservoir 180 can be subsequently discarded. The coated nanoparticles made using the frequency modulated flow reactor 100 are composed primarily of unoxidized iron, but may contain a small amount of iron oxide. In the case of iron nanoparticles coated with cPEG the small amount of iron oxide is thought to result from the reaction of the cPEG at the surface of the particle collection reservoir 160 and water absorbed to or near the surface of the nanoparticles.

Particles thus formed can be functionalized depending on the available groups of the coating. Procedures and methods for such functionalization are well known to those of skill in the art. Examples of functionalization techniques and reagents can be found in Lundblad and Noyes (1988) Chemical Reagents for Protein Modification, vols. 1-2, CRC Press, Inc., Boca Raton, Fla.; and Hugli (ed. 1989) Techniques in Protein Chemistry, Academic Press, San Diego, Calif., which are incorporated by reference in their entirety at least so to the extent that they disclose functionalization procedures.

A method of directing magnetic particles to a particular area of a patient includes making substantially unoxidized iron nanoparticles with a biocompatible coating, administering the particles to a patient (for example, in a saline solution), and adjusting a magnetic field proximate the patient to cause the particles to move from an administration site toward a desired area of the patient's body. The biocompatible coating can include polyethylene glycol (e.g., cPEG). In some cases, the biocompatible coating is functionalized.

The strong magnetic properties of the coated nanoparticles allow movement in a magnetic field gradient resulting in direct delivery of the functionalized particles to specific areas of the body, such as the brain. For example, nanoparticles can be delivered to the subarachnoid space by lumbar puncture and delivered to a specific portion of the body (such as the brain) with an MRI magnet.

Functionalized iron nanoparticles as described herein can be used for detecting specific target cells, affecting cell lysis of a target cell, and/or moving one or more cells. If a target cell has a specific agent that can be functionalized to the nanoparticles described herein the nanoparticles can be directed to the target cell. These functionalized nanoparticles can then be administered to a patient (e.g., in a saline solution). Localization of the particles can be, for example, detected by MRI. Alternatively, a magnetic field proximate the patient can be adjusted to cause the particles to heat up, causing an adjacent cell to die, i.e., cell lysis. Additionally, the targeted cell(s) can be moved from one location in the body to another by first attaching one or more iron nanoparticles to the cell(s) then causing the iron nanoparticles to move with a magnetic field.

Conjugation of a cell-specific compound to coated elemental iron nanoparticles allows for imaging before, during, and/or after therapy. Furthermore, coated iron nanoparticles exhibit a profound loss of magnetic resonance signal with TR or T2-weighted sequencing. Loss of magnetic resonance signal with the functionalized nanoparticles allows for precise magnetic resonance imaging of the particles in any area of the body into which the particles are delivered. For example, a sharp drop in the T2 weighted-image sequence of functionalized iron nanoparticles allows direct visualization of vascular structures and vascular pathology, including the coronary, carotid, and intracerebral circulation, by MRI. For example, coated iron nanoparticle bound to a viral protein or a virus allows MRI visualization during viral delivery for gene therapy applications. In another example, iron nanoparticles loaded with antibodies to cancer stem cells, such as anti-CD133, can be used to detect residual cancer cells after treatment.

Functionalized elemental iron nanoparticles are also suitable for hyperthermia treatment of cancer or other cells. The magnetic properties of these nanoparticles allow for localized hyperthermia applications by exposure to an oscillating magnetic field. By subjecting magnetic nanoparticles to an oscillating magnetic field, i.e., an alternating current (AC) field, heat is generated in a highly localized manner. Heat generation is the result of eddy current, hysteresis, and Brownian rotation loss mechanisms that all rely on the magnetic properties of the iron nanoparticles. See Rosensweig, R. E., J. Magnetism Magnetic Materials 2002, 252, 370-374.

Utilizing a magnetic field created by, for example, an MRI unit, these particles can be elevated in vivo to a temperature of at least 40° C. or higher in an oscillating magnetic field to lyse a cancer cell adjacent to, in the vicinity of, or coupled to the particle. Further examples of temperatures to which the particles can be raised include at least 41° C., at least 42° C., at least 43° C., and at least 44° C. A preferred temperature to which the particles can be raised is at least 43° C. The magnetic field can be, for example, a 40 e, 500 kHz AC magnetic field. The magnetization of the iron nanoparticles allows for heat dissipation at frequencies less than 1.2 MHz. Hysteresis loss is directly proportional to the area enclosed by the magnetization curve.

The iron nanoparticles described herein represent a new generation of magnetic nanoparticles which may serve as a multi-functional clinical tool. The term multi-functional is intended to indicate that the particles and methods described above can be used in various combinations, e.g., functionalization, imaging, and lysis, and is not intended to be limited to any particular combination or combinations which will be readily apparent to one of skill in the art. Based on their greater magnetization in comparison to conventional IONPs, these particles can serve as superior MRI contrast agents and can induce local hyperthermia. These elemental Fe-based nanoparticles achieve high magnetism without the introduction of other metal elements which may be biologically toxic. Furthermore, the biocompatible cPEG coating of the iron nanoparticles allows for covalent and stoichiometric attachment of peptides such as antibodies or receptor ligands specific to cancer cells providing surface functionalization and ultimate cell targeting. The magnetic targeting of specific tissues is also possible due to the superior magnetic properties of these nanoparticles.

### EXAMPLES

### Example 1

### Iron Nanoparticle Synthesis

Iron nanoparticles were synthesized by the chemical reduction of ferrous chloride with sodium borohydride in a reaction fitting (see equation I above) using an apparatus like the one described above and shown in FIG. 1. A 1.7-2.0 molar ratio of ferrous chloride to sodium borohydride was used to maximize the Fe content. Immediately upon synthesis, the nanoparticles are exposed to a bis-carboxyl terminated polyethylene glycol (cPEG) and ethanol solution to provide their biocompatible coating. Iron nanoparticles less than 10 nm in diameter were formed with a 2-3 nm thick cPEG coating as seen in FIGS. 3A-3D. The overall morphology of the nanoparticles synthesized is shown by the schematic in FIG. 3A. A highly uniform particle size distribution was obtained as seen in FIG. 3B for the low magnification bright field TEM image (using a JEM-3010FX TEM (JOEL; Tokyo, Japan)). The clustering of particles was due to the magnetism and surface tension of the sample droplet on the carbon coated TEM grid as the sample dried during preparation for imaging. An image of a single iron nanoparticle using high resolution TEM is shown in FIG. 3C. The iron nanoparticle shown in FIG. 3C contains multiple components as indicated by three distinct levels of contrast in the TEM image. As indicated in FIG. 3A, FIG. 3C shows a crystalline core that is surrounded by two amorphous shells (with less contrast as a function of radius). Given the nature of the synthesis, it is thought that the first core is Fe with interstitial boron and the outer shell is the cPEG coating.

The formation of borides using the borohydride reduction is dependent on the concentration of the reagents and since the concentrations are reduced as the reaction progresses, it is thought that more boron would be incorporated into the product as the nanoparticles nucleate and grow. See Glavee, G. N.; Klabunde, K. J.; Sorensen, C. M.; Hadjipanayis, G. C., Inorg, Chem. 1995, 34, 28-35. The selected area electron diffraction pattern (SAD) shown in FIG. 3D indicates there are no reflections from iron boride phases. However, there is evident peak broadening in the alpha Fe reflections. This can be attributed to either the nanoscale particle size, resulting from Scherer broadening, or to the presence of amorphous Fe component as boron is incorporated. We have found as the nanoparticle size increases, X-ray line broadening is maintained (due to an amorphous Fe-rich volume fraction which is present in the samples). Because boron is a grain refiner, it is plausible that the boron is simply straining the iron lattice. The lighter amorphous shell is thought to be due to reduced Z contrast resulting from the low molecular weight cPEG that coats the nanoparticles compared to the amorphous iron boron. The reaction between the carboxyl group on the cPEG and the nanoparticle surface incorporated a slight oxide layer. This layer accounts for the lighter reflections in the SAD pattern of FIG. 3D.

### Example 2

### Characterization of the Iron Nanoparticles of Example 1

### Magnetization

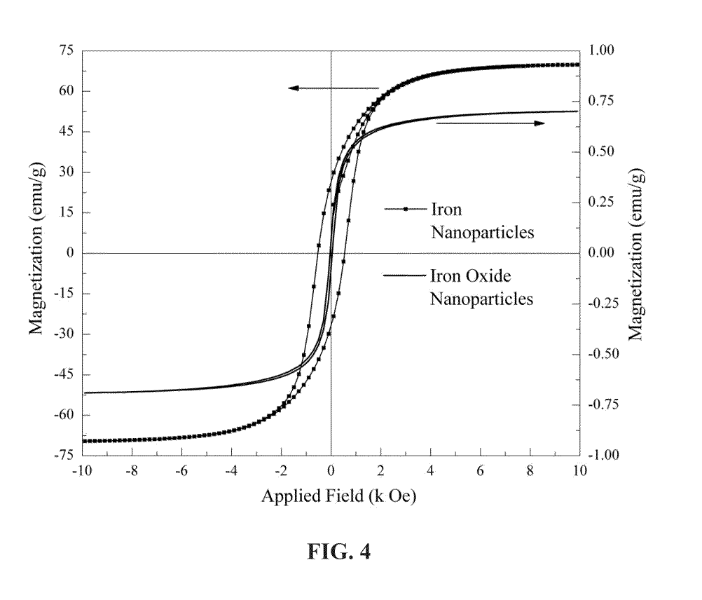

FIG. 4 shows magnetization curves of the iron nanoparticle made in Example 1 compared to oxidized-iron nanoparticles. The oxidized-iron nanoparticles used for comparison were Fe3O4 nanocrystals with 10 nm core size and coated with amphiphilic triblock polymers (2-3 nm) prepared as previously described. See Kim, M.; Chen, Y.; Liu, Y.; Peng, X., Adv. Mater. 2005, 17(11), 1429-1432; Gao, X.; Cui, Y.; Levenson, R. M.; Chung, L. W. K.; Nie, S., Nature Biotechnol. 2004, 22 (8), 969-976. The higher magnetization and soft ferromagnetic nature shown by the iron nanoparticles of Example 1 is evident from the magnetization curves shown in FIG. 4 in a comparison with the oxidized-iron nanoparticles. The saturation magnetization of 70 emu/g for the iron nanoparticles from Example 1 represents only a third of the bulk alpha-Fe value due to the nanoparticle size and the cPEG coating. In addition to greater magnetization, the coercivity for the iron nanoparticles is approximately 100 orders higher in comparison to oxidized-iron nanoparticles as shown in FIG. 4. The high magnetization and coercivity of the present particles make them ideal for biomedical applications such as MRI contrast enhancement and the induction of local hyperthermia for inducing cancer or other cell death.

### Heating

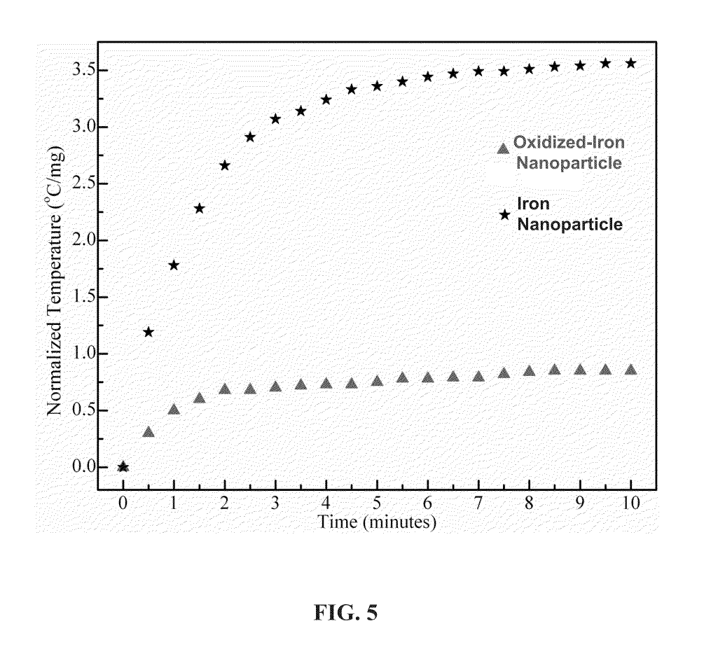

FIG. 5 shows the degree to which iron nanoparticles from Example 1 were heated upon exposure to an oscillating magnetic field. Both iron nanoparticles from Example 1 and oxidized-iron nanoparticles were dispersed in ethyl alcohol and excited with a 4 Oe 500 kHz magnetic field. As shown in FIG. 5, hysteresis losses are enhanced (i.e., normalized temperature is increased) in the iron nanoparticles from Example 1 as compared to the oxidized-iron nanoparticles. As can be seen in FIG. 5, there is a distinct difference in the achievable temperature per unit mass of the iron nanoparticle from Example 1 as compared to the oxidized-iron nanoparticles. As shown in FIG. 5, the increased magnetization afforded by the iron nanoparticles allowed for heat dissipation at frequencies in the safe range (<1.2 MHz) while the oxidized-iron nanoparticles would require much higher frequencies to facilitate the same thermal dose due to a higher FMR frequency. FIG. 5 shows that local hyperthermia is achievable with iron nanoparticles such as those made in Example 1, and that oxidized-iron nanoparticles would require a dose in excess of twenty-fold or greater to achieve the same temperature change.

### Imaging

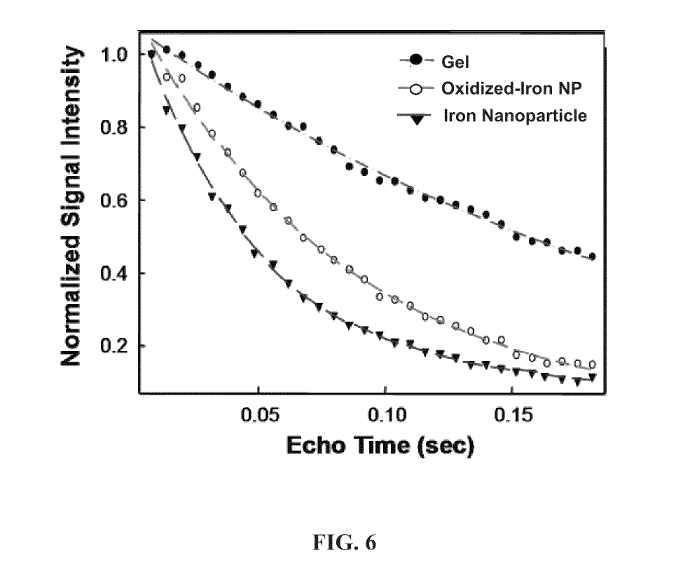

To assess imaging properties and the MRI contrast enhancing effect of the iron nanoparticles of Example 1, the longitudinal and transverse relaxivities, r1 and r2, were calculated from the measurement of T1 and T2 relaxation times of the iron nanoparticles at different concentrations at a magnetic field strength of 1.5 T on a clinical capable MRI scanner. For the MRI experiments, the iron nanoparticles from Example 1 or the oxidized-iron nanoparticles at given concentrations were suspended in a 1% agarose gel and placed in 10 mL tubes. For T1 measurement, an inversion recovery sequence was used with 9 non-equidistant different time delays (TI=0.1−1 s) between inversion and the first 90° excitation pulse. A non-linear curve fitting of the magnitude MRI signal intensity measured at different TI time points from ROIs selected in each sample to the equation of M(TI)=0×(1−C×exp(−TI/T1)), (where TI is inversion time and M is the magnitude of MRI signal), was used to calculate the T1 relaxation time of each sample. For T2 measurement, a multi-echo fast spin echo sequence was used to collect a series of data points simultaneously at different echo times (TEs of 6-180 ms with 6 ms increment). The T2 relaxation time of the iron nanoparticle sample was calculated by fitting the decay curve (FIG. 6) on a pixel-by-pixel basis using the non-linear mono-exponential algorithm of Mi=M0×exp(−TEi/T2). For the measurement of effective transverse relaxation time, T2*, multiple dual-echo gradient echo imaging sequences with susceptibility weighting was used. T2* values were obtained from the fit of the plot of MRI signal intensity versus echo times according to the equation: M(TE)=M(0)×exp(−TE/T2*)+C, where M(TE) is the signal intensity observed at a given echo time, TE, and C is a constant that reflects the background noise.

Table 1 summarizes the measurements of relaxivities r1, r2, and r2*, which are defined as relaxation rates R1, R2, and R2*, which are the reciprocal relaxation times (e.g., R1=1/T1), at the concentration unit and can be obtained from the slopes of the concentration vs. relaxation time plots. The data show that r2 and r2* relaxivity of the iron nanoparticles made in Example 1 is significantly higher than that of the oxidized-iron nanoparticles at comparable particle size. Such stronger T2 shortening effect, typically from stronger magnetic susceptibility, leads to spin dephasing and substantial MM signal drop.

TABLE 1
Relaxivities of Iron Nanoparticles and Oxidized-Iron Nanoparticles

| | Deionized H2O | Gd-DTPA | iron nanoparticles | oxidized-iron nanoparticles |
|---|---|---|---|---|
| r1 (s−1 · mM−1) | 0.01 ± 0.002 | 6.2 ± 0.24 | 1.2 ± 0.23 | 15.2 ± 1.1 |
| r2 (s−1 · mM−1) | 0.54 ± 0.12 | 8.2 ± 0.36 | 129 ± 5.9 | 56 ± 2.1 |
| r2* (s−1 · mM−1) | 0.43 ± 0.11 | — | 112 ± 3.4 | 54 ± 2.3 |

Currently most MRI exams, particularly clinical MRI, use magnetic resonance signals rising from protons of water that are abundant in the living systems. The image contrasts are mainly generated based on the endogenous differences of tissues or organs in the water content or in the magnetic relaxivities and susceptibility due to the different local or micro-environment of the tissue or organ where water molecules reside. Differences in relaxation times (e.g., T1 and T2) of water are typically exploited for generating imaging contrast to delineate the anatomical and morphological characteristics of the tissue or organs with specially designed RF pulse sequences. Exogenous agents can facilitate such differences to achieve the contrast enhancement as much as many orders of magnitudes. It is considered that the contrast enhancement and MRI signal amplification by superparamagnetic nanoparticles are mainly based on the magnetic susceptibility effect, i.e., T2 effect, and the local magnetic perturbations induced by the nanoparticles. Our results showed stronger T2 shortening effect from iron nanoparticles made in Example 1 than oxidized-iron nanoparticles which suggests that iron nanoparticles like those made in Example 1 have the potential to be more powerful contrast enhancing media than currently used oxidized-iron nanoparticles.

The particles, apparatus, and methods of the appended claims are not limited in scope by the specific particles, apparatus, and methods described herein, which are intended as illustrations of a few aspects of the claims and any particles, apparatus, and methods that are functionally equivalent are within the scope of this disclosure. Various modifications of the particles, apparatus, and methods in addition to those shown and described herein are intended to fall within the scope of the appended claims. Further, while only certain representative combinations of the particles, apparatus components, or method steps disclosed herein are specifically described, other combinations of the method steps or combinations of elements of a composition are intended to fall within the scope of the appended claims. Thus a combination of steps, elements, or components may be explicitly mentioned herein; however, all other combinations of steps, elements, and components are included, even though not explicitly stated.

The term comprising and the variations thereof as used herein are used synonymously with the term including and variations thereof and are open, non-limiting terms.

What is claimed is:

1. A conjugate particle comprising a substantially unoxidized iron core, a biocompatible coating, and a cell-specific agent.

2. The particle of claim 1, wherein the cell-specific agent is a cancer cell specific agent.

3. The particle of claim 1, wherein the cell-specific agent is a polypeptide or an antibody.

4. The particle of claim 2, wherein the cell-specific agent is an antibody to VEGF, PDGF receptor, EGF receptor, or truncated viii EGF receptor.

5. The particle of claim 1, wherein the biocompatible coating comprises polyethylene glycol.

6. A method for detecting specific target cells comprising:
providing particles comprising substantially unoxidized iron cores, and biocompatible coatings functionalized with a cell specific agent that has an affinity for the target cells;
administering the particles to a patient; and
detecting localization of the particles by magnetic resonance imaging.

7. The method of claim 6, wherein the target cells are cancer cells.

8. The method of claim 6, wherein the cell specific agent is a cancer-cell specific agent.

9. The method of claim 8, wherein the cancer-cell-specific agent is a polypeptide or an antibody.

10. The method of claim 6, wherein the cell-specific agent is an antibody to VEGF, PDGF receptor, EGF receptor, or truncated vIII EGF receptor.

11. The method of claim 6, wherein the biocompatible coating comprises polyethylene glycol.

12. A method for lysis of a target cell comprising:
providing particles comprising: substantially unoxidized iron cores, and biocompatible coatings functionalized with a cell-specific agent that has an affinity for the target cells;
administering the particles to a patient; and
adjusting magnetic fields proximate the patient to cause cell lysis of the target cell.

13. The method of claim 12, wherein the magnetic field is an oscillating magnetic field.

14. The method of claim 12, wherein the particles have FMR of less than 1.2 MHz using 500 MHz and 4 Oe AC magnetic field.

15. The method of claim 12, wherein the particles area heated to at least 43° C. in vivo.

16. The method of claim 12, wherein the cell specific agent is a cancer-cell-specific agent.

17. The method of claim 16, wherein the cancer specific agent is a polypeptide or an antibody.

18. The method of claim 12, wherein the cell-specific agent is an antibody to VEGF, PDGF receptor, EGF receptor, or truncated vIII EGF receptor.

19. The method of claim 12, wherein the biocompatible coating comprises polyethylene glycol.
